(19) United States
(12) Patent Application Publication
(10) Pub. No.: US 2022/0106634 A1
(43) Pub. Date: Apr. 7, 2022

(54) DRY REAGENT, REAGENT KIT, METHOD OF PRODUCING DRY REAGENT, ANALYTICAL METHOD AND NUCLEIC ACID AMPLIFICATION METHOD

(71) Applicant: Canon Medical Systems Corporation, Otawara-shi (JP)

(72) Inventor: Naoshige IZUMIKAWA, Kawasaki (JP)

(73) Assignee: Canon Medical Systems Corporation, Otawara-shi (JP)

(21) Appl. No.: 17/493,123

(22) Filed: Oct. 4, 2021

(30) Foreign Application Priority Data
Oct. 5, 2020 (JP) 2020-168506

(51) Int. Cl. C12Q 1/6848

(52) U.S. Cl. CPC C12Q 1/6848

(57) ABSTRACT

According to one embodiment, a dry reagent includes an insoluble fiber and a dried material containing a biological material, which is retained in the fiber.

### CROSS-REFERENCE TO RELATED APPLICATIONS

This application is based upon and claims the benefit of priority from Japanese Patent Application No. 2020-168506, filed Oct. 5, 2020, the entire contents of which are incorporated herein by reference.

### FIELD

Embodiments described herein relate generally to a dry reagent, a reagent kit, a method for producing the dry reagent, an analytical method, and a nucleic acid amplification method.

### BACKGROUND

In order to freeze-dry and store proteins in a stable state, various carbohydrates and polymers have been used. Of these, high-molecular sugars contribute to the stability of protein in the storage after drying.

However, high-molecular sugars, if concentrated by freeze-drying, become highly viscous during storage or use as they absorb moisture in the air. Therefore, when a dried material is re-dissolved into a solvent to be used, the re-solubility may be lowered.

### BRIEF DESCRIPTION OF THE DRAWINGS

FIG. 1 is a diagram showing a dry reagent according to an embodiment, which includes a part (a) showing the reagent in its entirely and a part (b) showing an enlarged view thereof.

FIG. 2 is a diagram showing the dry reagent for the embodiment.

FIG. 3 is a flowchart illustrating a production method for the dry reagent according to the embodiment.

FIG. 4 is a schematic diagram showing the production method for the dry reagent according to the embodiment.

FIG. 5 is a schematic diagram showing how to use the dry reagent according to the embodiment.

FIG. 6 is an enlarged view of the dry reagent of the embodiment in storage.

FIG. 7 is a flowchart of a nucleic acid amplification method according to an embodiment.

### DETAILED DESCRIPTION

In general, according to one embodiment, a dry reagent comprises an insoluble fiber and a dried material containing a biological material, which is retained in the fiber.

Embodiments will be described hereinafter with reference to the accompanying drawings.

### First Embodiment

Dry Reagent

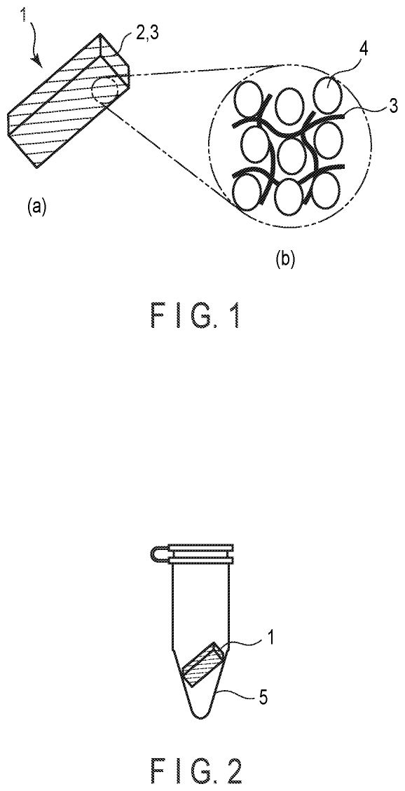

As shown in FIG. 1, parts (a) and (b) a dry reagent 1 according to an embodiment comprises a dry object material 2 and fibers 3 that holds the dry object material 2. The dry object material 2 contains biological materials 4. The fibers 3 function as a scaffold for the dry object material 2. As will be described in detail later, the dry reagent 1 of the embodiment is made into a dry form in order to store the dry object material 2 for a certain period of time, and when the dry object material 2 is to be used, for example, the dry reagent 1 is brought into contact with an appropriate liquid (hereinafter also referred to as “second solution”) to resolve the dry object material 2, to remove the fibers 3. Thus, the dry object material 2 can be used for a desired application.

The biological materials 4 should preferably be a protein or peptide, for example. Or, the dry object material 2 may be nucleic acid, for example, DNA or RNA, bacteria, fungi, virus or the like. The dry object material 2 contained in one dry reagent 1 may contain multiple kinds of biological materials 4.

The dry object material 2 may contain a further component in addition to the biological materials 4. For example, the dried product 2 may further contain, for example, a salt, a thickener, a preservative and/or a buffer for pH preparation, though particularly not limited.

The fibers 3 are, for example, a structure formed by aggregation of multiple fibers 3. For example, the fibers 3 are intertwined with each other and there are gaps between the fibers 3. The fibers 3 are, for example, a plurality of fibers 3 formed into a woven fabric, a non-woven fabric, or a bundle.

The components contained in the dry object material 2, for example, the biological materials 4, are held in the gaps between the fibers 3 or on the surfaces of the fibers 3, as shown in FIG. 1, part (b).

The fibers 3 are insoluble fibers 3.

The fibers 3 should preferably be hydrophilic to the extent that it can maintain its own structure in the second solution when resolved. With the hydrophilicity, it is possible to improve the re-solubility. Further, the fibers 3 should preferably be those interacting appropriately with the biological materials 4. The term “appropriate interaction” means, for example, interaction to the extent that the biological materials 4 are held between the fibers 3 without losing the activity of the biological materials by the bonding with the fibers 3.

As such fibers 3, for example, cellulose fibers or hydrophilic treated glass fibers can be used.

As the fibers 3, any of the above-listed fibers 3 commercially available can be used. As the commercially available fibers 3, a cellulose fiber pad or glass fiber pad can be used. For example, the commercially available fibers 3 can be processed into a desired size to be used, or multiple types of commercially available fibers 3 can be used in combination.

The overall shape of the fibers 3 may be, for example, rectangular parallelpiped, cubic, etc., and may also be spherical or sheet-like, but is not limited thereto.

Although not shown in the figure, the dry reagent 1 may further contain low molecular weight sugar and/or surfactant.

The low-molecular-weight sugar is the type which does not impair the activity of the biological materials 4, and should preferably be a monosaccharide or disaccharide. As the low-molecular-weight sugar, for example, glucose, fructose, galactose, sucrose, maltose, or lactose can be used.

The surfactant is the type which does not impair the activity of the biological materials 4, and for example, a nonionic surfactant can be used.

In a further embodiment, as shown in FIG. 2, the dry reagent 1 may be contained in a container 5 to be provided.

As the container 5, for example, a microtube, test tube, centrifuge tube or aluminum- or plastic-made bag can be used. By storing in a container 5, it is easier to manage the dry reagent 1 when it is stored, and also, as will be described in detail later, it is possible to add a solution (the second solution) to the container 5 for re-dissolution. Thus, when, for example, aseptic re-dissolution is required, the procedure can be simplified.

Production Method

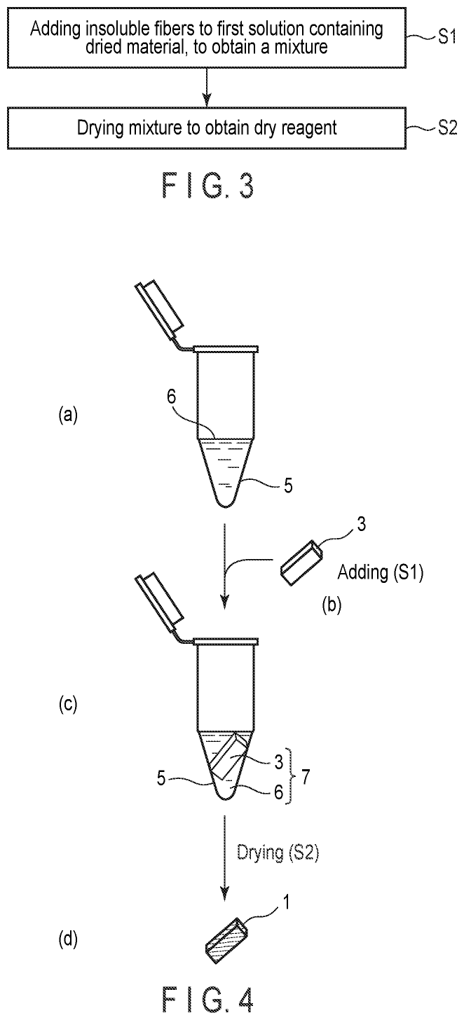

Next, a method of producing the dry reagent of the embodiment will be described. As shown in FIG. 3, the production method includes an addition step (S1) of adding the fibers 3 to a solution 7 (hereinafter also referred to as “first solution”) containing the dry object material 2, to obtain a mixture, and a drying step (S2) of drying the mixture to obtain a dry reagent.

An example of the production method will now be described in detail below with reference to FIG. 4.

First, as shown in FIG. 4, part (a), a first solution 6 is prepared. The first solution 6 can be obtained, for example, by dissolving the dry object material 2 containing desired components into an appropriate solvent. The appropriate solvent is a liquid whose effect on the activity of the dry object material 2 is within a negligible degree and which be removed by drying. The solvent can be, for example, water, buffer solution, saline solution or the like.

The amount of the first solution 6 to be used is selected, for example, according to the amount of biological material 4 to be used at a time. For example, the first solution 6 for producing one dry reagent 1 contains the amount of biological material 4 used for one experiment. The first solution 6 can be prepared, for example, in the container 5 as described above.

As shown in FIG. 4, part (b), the fibers 3 are prepared. The amount of the fiber 3 to be used is selected according to the amount of the first solution 6.

Next, as shown in FIG. 4, part (c), the fibers 3 are added to the first solution 6 to obtain a mixture 7 (addition step (S1)). The addition step (S1) is carried out, for example, by immersing the fibers 3 into the first solution 6. For example, the fibers 3 may be completely immersed in the first solution 6. The addition step (S1) may also be carried out, for example, by dropping the first solution 6 on the fibers 3.

After immersing the fibers 3 in the first solution 6, further agitation and leaving it to stand still, etc. may be carried out so as for the first solution 6 to be absorbed to the interiors of the fibers 3.

In the case where the low-molecular sugar and/or surfactants described above are used, they may be added to the first solution 6 before the addition step (S1), or they may be added to the mixture 7 after the addition step (S1).

Next, as shown in FIG. 4, part (d), the mixture obtained in the step (S1) is dried to obtain the dry reagent 1 (drying step (S2)).

The drying step (S2) is carried out, for example, by warm air drying or freeze drying.

In the case of using warm air drying, for example, it can be carried out using a dryer. For example, the mixture 7 is placed in a drying chamber of the dryer, and the moisture is removed from the mixture 7 by sending dry warm air to the mixture 7. The conditions should be such that, for example, the temperature should be near such a room temperature that the biological materials 4 are not denatured, for 20 to 180 hours. The warm air drying may as well be carried out under vacuum conditions. In the case of the warm air drying at high temperature, it is preferable to dry with hot air at 90 to 100° C. for 5 to 10 minutes.

In the case of the freeze drying, for example, the mixture 7 is pre-frozen with liquid nitrogen or the like, and the pressure is reduced to about 10 to 20 Pa in a freeze-dryer, flowed by drying −40 to −80° C. The freeze drying can be carried out, for example, using a commercially available freeze dryer.

The drying process may be carried out on the mixture 7 while in the container 5, or onto the mixture 7 as removed from the container 5.

The dry reagent 1 obtained by step (S2) may be stored in the container 5 used in the production, or may be removed from the container 5 as shown in FIG. 4, part (d). In that case, it may be stored in some other container.

Analysis Method

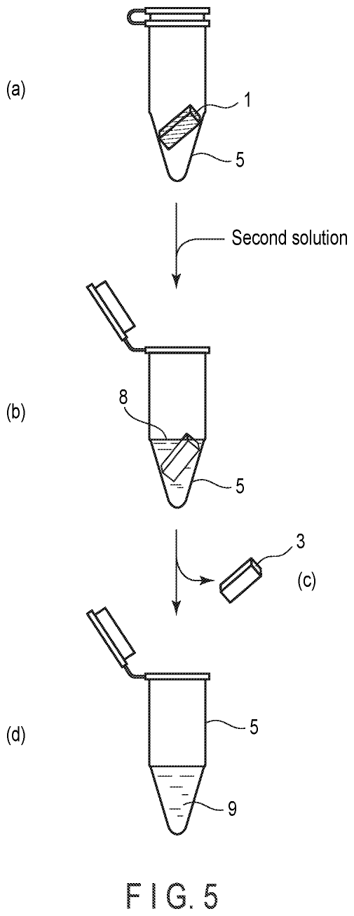

The analysis method using the dry reagent of the embodiment will be described using FIG. 5.

First, as shown in FIG. 5, part (a), the dry reagent 1 is stored in the container 5. Here, as shown in FIG. 2, the dry reagent 1 while pre-contained in the container 5 may be used as it is.

Next, as shown in FIG. 5, part (b), a second solution 8 is added to the container 5. The second solution 8 is used to resolve components, for example, the biological materials 4, contained in the dry object material 2 of the dry reagent 1, and is, for example, water, buffer solution, saline solution, or the like.

After that, agitation may then be carried out to detach the dry object material 2 from the fibers 3 and dissolve it into the second solution 8. The agitation may be carried out by pipetting, tumble mixing or vortexing.

With such redissolution, a liquid reagent 9 can be obtained, in which the dry object material 2 is dissolved into the second solution 8. At this time, the fibers 3 are insoluble and do not dissolve into the second solution 8, remaining as a solid material.

Next, the fibers 3 are removed from the liquid reagent 9. For example, the liquid reagent 9 can be separated using a micropipette or the like and stored in a separate container. Alternatively, as shown in FIG. 5, parts (c) and (d), the fibers 3 may be removed from the container 5 using tweezers or the like, and the liquid reagent 9 may be left in the container 5.

In the above-described manner, the liquid reagent 9 which includes the dry object material 2 contained in the dry reagent 1 can be obtained. The liquid reagent 9 can be used in a desired next step of the analytical method. The next steps, though not particularly limited to the following, include a biological, biochemical and/or physiological analysis to carried out using the biological material 4, for example. The following process, though not particularly limited to the following, include, for example, nucleic acid amplification, nucleic acid detection, immunoassay, western blotting, flow cytometry and/or microscopy, described in the second embodiment.

In the dry reagents according to the embodiments described above, the fibers are used as a scaffold for the dried material, and therefore the reagents are highly re-dissolvable. For example, in the conventional methods, high-molecular sugars are used as the scaffold; however high-molecular sugars easily absorb moisture in the air during storage. Thus, a highly viscous film can be formed around the dried material, thereby reducing the re-solubility of the dried material.

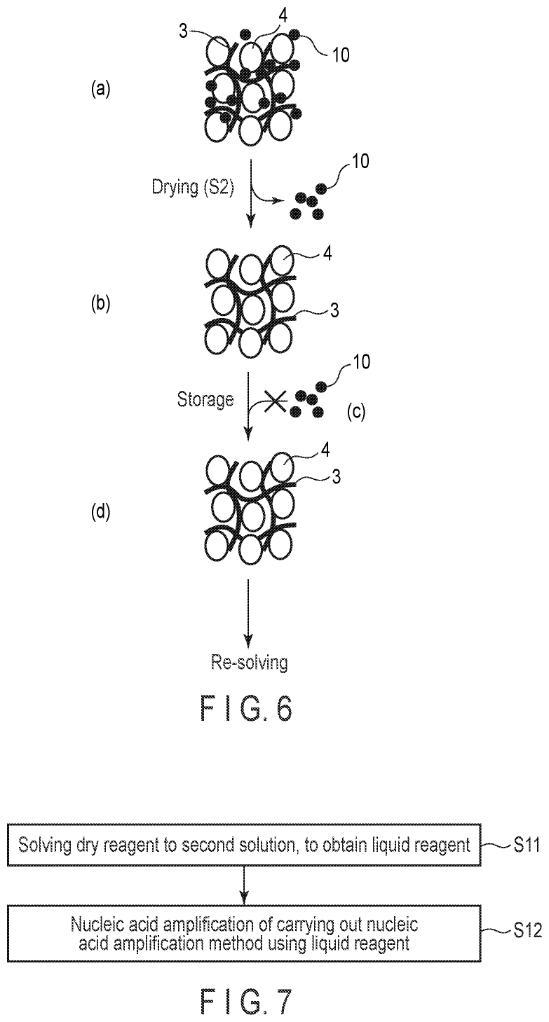

According to the dry reagent of the embodiment, for example, as shown in FIG. 6, parts (a) and (b), even if the reagent is stored while water molecules 10 exist even after water molecules 10 attached to the biological materials 4 and the fibers 3 are subjected to evaporation by the drying step (S2), water molecules 10 are not substantially absorbed to the biological materials 4 and the fibers 3 as shown in FIG. 6, part (c). As a result, as shown in FIG. 6, part (d), even after storage, the dry reagent 1 does not contain water molecules 10 and is easily dissolved into liquid. Therefore, the dry reagent of the embodiment has high re-solubility. Thus, the work and time to use in the following step can be omitted, and the activity of the biological material can be easily exhibited.

Further, the fibers are used as a scaffold for the dried materials, and thus it is possible to prevent the biological materials from aggregating with each other as they are placed between the fibers, making it possible to dry and store the biological materials in a stable manner while retaining their functions. The function-retaining state means that the three-dimensional structure of the biological material does not change significantly before and after drying, and maintains its original activity and properties. Therefore, the fibers act as a stabilizer for the biological material during drying and storage.

Further, when drying a dried material in a liquid state, it is conventionally necessary to use vacuum drying to prevent the liquid from dispersing due to boiling. On the other hand, the dry reagent of the embodiment is subjected to the drying process while the first solution absorbed into the fibers, thus preventing the liquid contained in the first solution from dispersing by boiling. Therefore, the dry reagent of the embodiment can be produced not only by vacuum drying but also by warm air drying. Therefore, an expensive vacuum dryer is not necessary, and the product can be manufactured at a low cost.

Further, the use of hydrophilic fibers and the low-molecular sugar and/or surfactant contained therein, the re-solubility of the dry object material 2 can be further enhanced.

Reagent Kit

According to a further embodiment, a reagent kit containing a dry reagent 1 is provided. The reagent kit includes the dry reagent 1 and the second solution.

For example, the dry reagent 1 and the second solution are contained in separate containers to be provided.

The reagent kit may further include, for example, a reagent necessary for the processing step to be performed after the dry reagent 1 is resolved.

### Second Embodiment

In the second embodiment, the dry reagent includes an enzyme used for nucleic acid amplification as a biological material. The rest of the configuration is similar to that of the first embodiment, and can be produced by the method shown in FIG. 3.

The enzyme used for nucleic acid amplification is, for example, DNA polymerase, RNA polymerase, or reverse transcriptase. The type of enzyme is selected according to the type of amplification method used in the nucleic acid amplification process, which will be described below. One dry reagent contains, for example, an amount of enzyme used for one nucleic acid amplification.

Next, the method of performing nucleic acid amplification using the dry reagent according to the second embodiment above will be described. As shown in FIG. 7, the method comprises a resolving step (S11) of resolving the dry reagent into the second solution, thereby obtaining a liquid reagent, and a nucleic acid amplification step (S12) of carrying out nucleic acid amplification using the resolved liquid reagent.

The resolving step (S11) can be carried out in a manner similar to that of the resolution described in the use method in the first embodiment.

Between the resolving step (S11) and the nucleic acid amplification step (S12), a step of removing the fibers 3 from the liquid reagent 9 thus obtained may be inserted. For example, the fibers 3 may be removed from the liquid reagent 9, or the liquid reagent 9 may be transferred to another container.

The second solution 8 may contain some other reagents to be used for nucleic acid amplification. Alternatively, some other reagents used for nucleic acid amplification may be added to the liquid reagent 9 immediately before the nucleic acid amplification step. The other reagents are, for example, primer sets, salts, deoxynucleoside triphosphates (dNTPs), thickeners, buffers for pH preparation, surfactants and/or ions. These other reagents may be contained in the dry reagent in advance.

Next, the nucleic acid amplification is carried out (the nucleic acid amplification step (S12)).

The nucleic acid amplification step may be carried out by a nucleic acid amplification method in which temperature is varied, such as the PCR method, or an isothermal nucleic acid amplification method such as the LAMP method, or a reverse transcription reaction may be performed before to these amplification reactions.

According to the dry reagent of the second embodiment described above, during storage, water molecules are not absorbed into the biological materials and fibers, and therefore the biological materials are highly resoluble. Thus, the nucleic acid amplification reaction can be carried out efficiently.

The dry reagent of the second embodiment may also be provided as a reagent kit that further includes a second solution. The reagent kit may further contain, for example, the other reagents as described above necessary for the nucleic acid amplification process and/or reagents necessary for the detection of the amplification product, such as a reagent containing a dye for optical detection or a reagent having an electric charge for electrical detection.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A dry reagent comprising:
an insoluble fiber; and
a dried material containing a biological material, which is retained in the fiber.

2. The dry reagent of claim 1, wherein
the biological material is a protein or peptide.

3. The dry reagent of claim 1, wherein
the biological material is an enzyme used for nucleic acid amplification.

4. The dry reagent of claim 1, wherein
the fiber is hydrophilic.

5. The dry reagent of claim 1, wherein
the fiber is a glass fiber or a cellulose fiber.

6. The dry reagent of claim 1, further comprising a low-molecular sugar and/or a surfactant.

7. The dry reagent of claim 1, wherein
the dry reagent is used as a liquid reagent by resolving it in a solution to remove the fiber.

8. A reagent kit comprising:
a dry reagent according to claim 1; and
a second solution which resolve the dry reagent.

9. A method for producing a dry reagent, comprising:
adding an insoluble fiber to a first solution containing a dried material containing a biological material, to obtain a mixture; and
drying the mixture to obtain the dry reagent.

10. The method of claim 9, wherein
the drying is carried out by warm air drying or freeze drying.

11. The method of claim 9, wherein
the biological material is a protein or peptide.

12. The method of claim 9, wherein
the fiber is a glass fiber or cellulose fiber.

13. The method of claim 9, wherein
the adding comprising further adding a low-molecular sugar and/or a surfactant to the first solution.

14. The method of claim 9, wherein
the dry reagent is used as a liquid reagent by resolving it in the solution to remove the fiber.

15. An analytical method comprising:
dissolving a dry reagent containing an insoluble fiber and a dried material containing a biomolecule, which is retained in the fiber, into a second solution to obtain a liquid reagent.

16. The analytical method of claim 15, further comprising:
removing the fiber from the liquid reagent after the dissolving.

17. A nucleic acid amplification method comprising:
re-dissolving a dry reagent containing an insoluble fiber and a dried material containing an enzyme to be used for nucleic acid amplification, which is retained in the fiber, into a second solution to obtain a liquid reagent; and
carrying out nucleic acid amplification using the liquid reagent.

18. The nucleic acid amplification method of claim 17, further comprising:
removing the fiber from the liquid reagent after the re-dissolving.
